(19) United States
(12) Patent Application Publication
(10) Pub. No.: US 2012/0215095 A1
(43) Pub. Date: Aug. 23, 2012

(54) X-Ray radiation reduction system

(76) Inventors: Amit Mordechai Av-Shalom, Vancouver (CA); Meir Deutsch, Vancouver, CA (US); Daniel Gelbart, Vancouver (CA)

(21) Appl. No.: 13/385,402

(22) Filed: Feb. 21, 2012

Related U.S. Application Data
(60) Provisional application No. 61/457,298, filed on Feb. 22, 2011.

(51) Int. Cl. A61B 6/00; A61B 6/08

(52) U.S. Cl. 600/424; 378/98.9

(57) ABSTRACT

By exposing the ROI at full exposure and full frame rate while exposing the area outside the ROI with low exposure and up to full frame rate an overall reduction in X-Ray radiation is achieved. The resultant image has slightly lower resolution outside the ROI but better resolution (as compared to standard fluoroscopy practices) in the ROI because of reduced scattering. Different exposures are supplied to different parts of the image by using a fast shutter in conjunction with the exposure control.

### FIELD OF THE INVENTION

The invention relates to the medical field and in particular to the art of continuous X-Ray procedures such as fluoroscopy.

### BACKGROUND OF THE INVENTION

The increased use of minimally invasive surgery caused an increase in the use of fluoroscopy, exposing the patients, doctors and support staff to ever increasing amounts of radiation.

A typical fluoroscopy unit includes a frame holding the X-Ray source and detector, a patient on a bed and a workstation. The portable units are known as “C-arm” units because of the C-shaped frame. Existing fluoroscopy systems expose a certain field-of-view (FOV) defined by the setting the collimator blades. The physician performing the fluoroscopy is usually interested in a smaller region-of-interest (ROI) within the FOV, however the larger image is required for orientation and periodic monitoring. Modern fluoroscopy machines use flat panel detectors and pulsed X-ray tube operation. Current generation fluoroscopy machines determine the x-ray tube current and pulse length automatically while the pulse's frequency is left under operator's control. This is illustrated in FIG. 1 in which all x-ray pulses expose the full FOV.

Prior art for reducing total radiation without limiting the viewing area is disclosed in U.S. Pat. No. 7,983,391 which adds a fast moving shutter that can set a different exposure area for every X-ray exposure pulse. Using this shutter, the system can expose the small ROI for few consecutive pulses and open up the shutter to the full collimator FOV for a single pulse, as shown in FIG. 2. For most of the pulses the shutter is partially closed, allowing only a small ROI to be exposed. Periodically the shutter is opened for one pulse to update the background image. Full details are given is the above mentioned patent. Image blending software that runs on the machine's workstation blends the ROI (which is a “live” sequence) with the surrounding taken during the full-FOV exposure pulse. The shutter can be placed anywhere in the X-ray beam path, but the preferred location is between the X-ray tube and the collimator in order to minimize the size of the shutter. The concept from U.S. Pat. No. 7,983,391 works well in procedures performed on moving body parts, such as the brain (for example during brain aneurysm coil embolization). For areas with rapid change, such as cardiac procedures, it will not capture all the motion in the area outside the ROI. The current invention overcomes this problem.

### SUMMARY OF THE INVENTION

By exposing the ROI at full exposure and full frame rate while exposing the area outside the ROI with low exposure and up to full frame rate an overall reduction in X-Ray radiation is achieved. The resultant image has slightly lower resolution outside the ROI but better resolution (as compared to standard fluoroscopy practices) in the ROI because of reduced scattering. Different exposures are supplied to different parts of the image by using a fast shutter in conjunction with the exposure control.

### BRIEF DESCRIPTION OF THE DRAWINGS

FIG. 1 is a schematic depiction of a series of images acquired using current fluoroscopy practices.

FIG. 2 is a schematic depiction of a series of images acquired using the method of U.S. Pat. No. 7,983,391.

FIG. 3 is a schematic depiction of a series of images acquired using the method of the invention.

FIG. 4 is a block diagram of a fluoroscopy system incorporating the invention.

FIG. 5 is a plan view of a shutter suitable for the invention.

FIG. 6 is an example of an X-Ray image where the ROI was exposed at a higher dose.

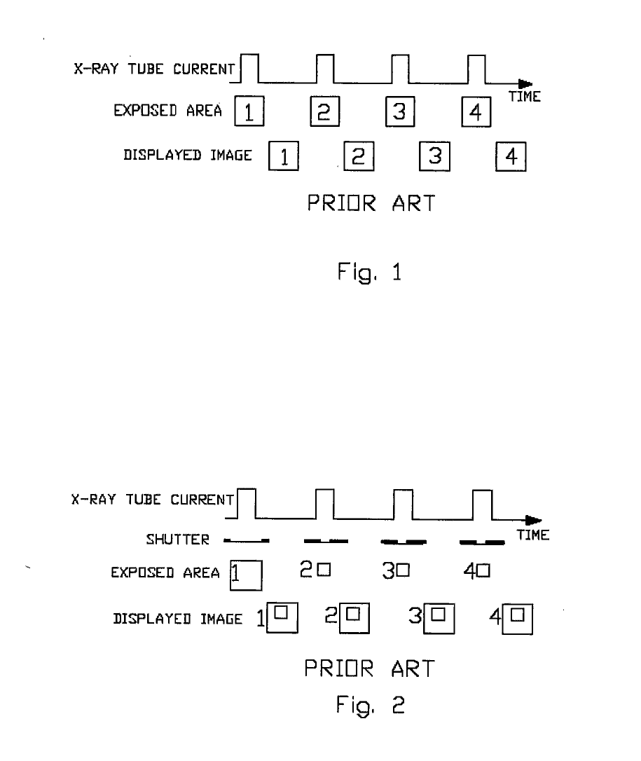

### DETAILED DISCLOSURE

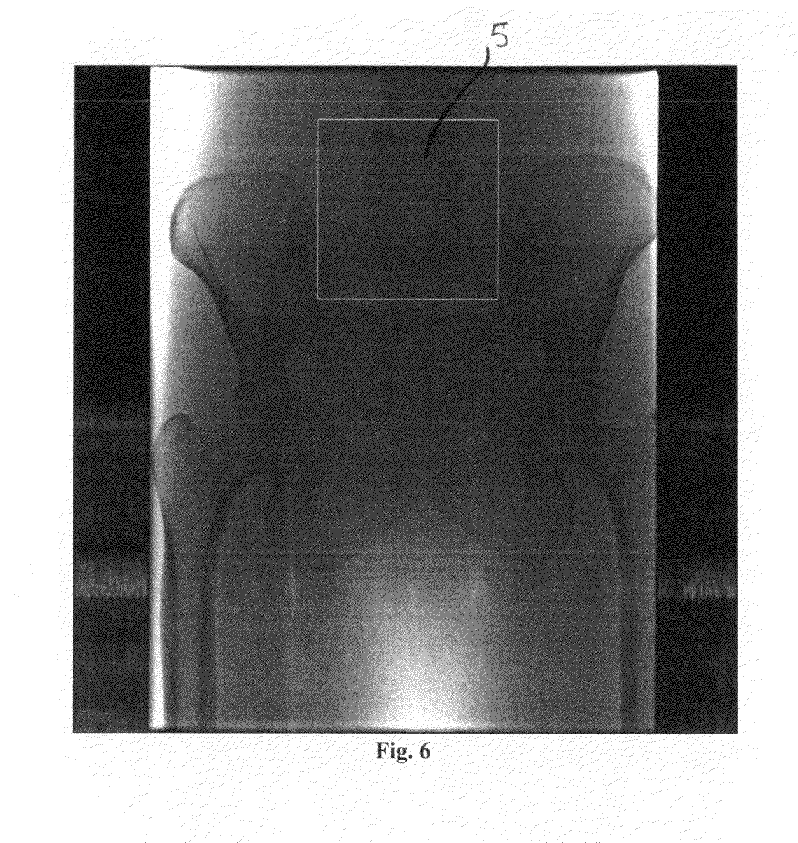

This application is an improvement on U.S. Pat. No. 7,983,391 which is hereby incorporated by reference in its entirety. The improvement allows a use of a shutter controlled ROI to be applied to procedure having fast moving body organs outside the ROI, such as the beating heart during cardiac interventions. The area outside the ROI is imaged at a lower exposure dose, achieved by lowering the X-ray tube current, pulse width or the tube voltage. Since changing the voltage alters the energy distribution of the beam, it is desired to keep the voltage constant and lower the pulse width and current. This assumes that a substantial reduction in dose (for example 10 fold reduction) can be achieved by a combination of lower current and shorter pulse and still produce a reasonable image quality, sufficient for the physician to watch the non-ROI area of the image. We performed an initial experiment to validate this assumption. We exposed a human-body phantom model twice to x-ray at the same voltage (“KV” setting) but with an order of magnitude different exposure and combined the two images by software. The result is shown in FIG. 6. The area inside the ROI 5 was exposed at full dose and combined with the background image.

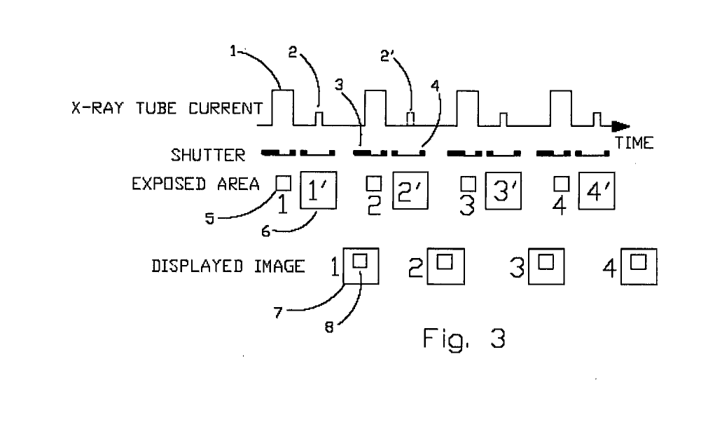

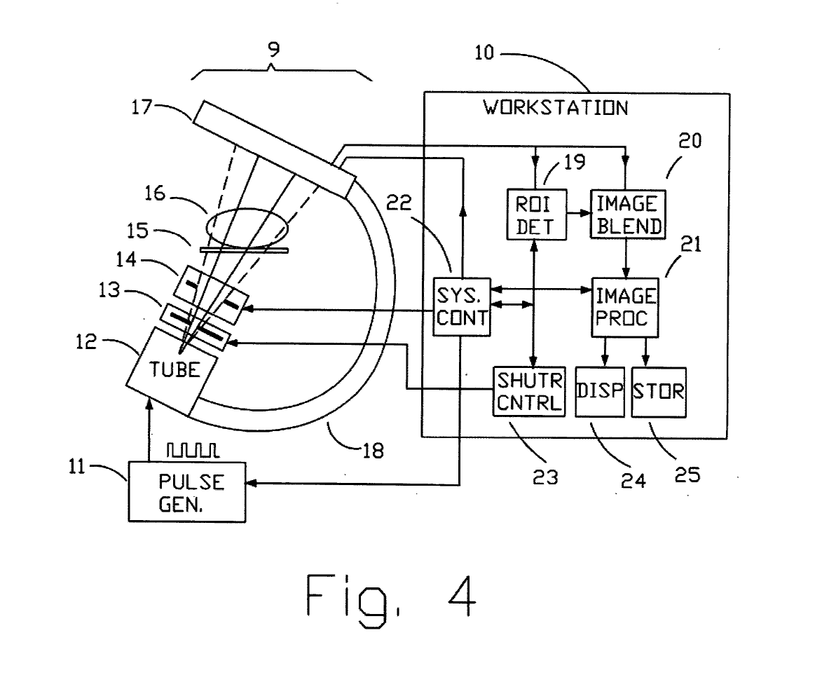

Referring now to FIG. 3, a sequence of full intensity X-Ray pulses 1 is limited to expose only a small ROI 5 by the action of a fast shutter having a limited opening 3. In between pulses 1 a lower dose pulse 2 is inserted and the shutter is momentarily opened to position 4, allowing the full FOV to be exposed. Pulse 2 does not need to be inserted after each pulse 1; for areas with slower change some of the lower intensity pulses can be completely omitted, as shown by missing pulse 2′. The high quality image 5 is blended with the background image 6 to form a composite image 7 in which the ROI 8 is exposed at a higher radiation dose than the background, which is the area outside the ROI. The lower dose images can be used to further improved the ROI image or can be discarded inside the ROI area. The area outside the ROI will appear slightly more noisy than a conventional image while the ROI area will be sharper than a conventional image as the narrow collimation angle reduces scatter. In FIG. 6 the ROI has an exposure dose about 10 times more that the full FOV image. Total radiation reduction in this case is about 5.5 fold assuming the ROI in this example is about 1/12 of the total area. Without the invention, using 10 exposures per second as an example, the conventional way will produce the accumulated radiation of 10 images per second while by using the invention the accumulated radiation per second will be 10/12+10× 1/10=1.83 images, giving an improvement of 10/1.83 or approximately 5.5 times.

Referring now to FIG. 4, a typical fluoroscopy system comprises of a C-arm assembly 9 and a workstation 10. The patient 16 is placed on a bed 15 between the X-Ray tube 12 and detector 17, typically a flat panel solid state detector. A fast shutter 13 is inserted between the X-ray tube 12 and the existing collimator 14. The collimator 14 is used to set the full FOV the conventional way, the shutter 13 defines the area of interest according to a manual or automatic setting. An automatic setting can be based on image recognition, tool recognition (such as tip of catheter or stent), motion analysis or any one of the many method used in computer vision. A manual setting can be based on any contact or non-contact input device, including touch screens, speech recognition and eyeball tracking. Recently interfaces capable of recognizing hand gesturing became available (such as Microsoft Kinect) which could be very suitable for defining an ROI without contact. Non contact interfaces are desirable, of course, to preserve sterility in the operating room.

The different dose pulses are generated by pulse generator 11 controlled by workstation 10. The main functional blocks in the workstation are ROI detector 19, Image Blending 20, Image Processing 21, System Control 22, Shutter Control 23, Display 24 and storage device 25. Most of these blocks are implemented in software. The only modules that do not exist in a standard fluoroscopy system are 13, 19, 20 and 23. The image blending can be simply by using a soft transition between the two regions, aided by the natural blur zone of about 10-20 mm created by the finite size of the X-Ray source. System geometry defines the blur zone. A simpler method is to detect the boundary of the ROI by setting a threshold on the data acquired with the shutter closed. Such a threshold can be set, by the way of example, at 50% of the peak detector signal. Any area below the threshold (but not inside the ROI) is discarded and replaced by the background image. The advantage of this simplified method is that the transition zone is only a single pixel and may be used without image blending software. Another alternative to blending the ROI into the full image is to place a visible border around the ROI, denoting to the user the high resolution area. This can be seen in FIG. 6. Such a border masks the undesired border formed by imperfect blending.

Typical distance between the fast shutter 13 and the X-Ray point source is 50-100 mm. Typical source size is 0.5-1.5 mm. The Shutter Control activates the shutter blades to form an opening of the size and location determined by the ROI detector 19. Note that current generation fluoroscopy machines include an AEC (Automatic Exposure Control) mechanism that analyzes the image from the detector and adjusts the x-ray tube parameters to achieve optimal image quality, as determined by the machine preset manufacturer tables. An important part of the solution depicted in FIG. 4 is to synchronize this AEC mechanism to the strong-weak pulse-pair, so the machine won't react as a result of the lower image quality that results from the lower dose exposure during the added x-ray pulse.

The preferred embodiment uses an electromagnetic actuator to move the shutter blades. Other actuators, such as pneumatic or hydraulic, can be used as well. An X-ray opaque liquid, such as Angiography dyes, can also be fed between two X-ray transparent plates to serve as an actuator blade. The preferred actuator design is similar to the one used in computer disc drives (“hard drives”). This actuator has a fast response time of about 10 mS, low cost and high reliability. Since it is well known no further details are needed.

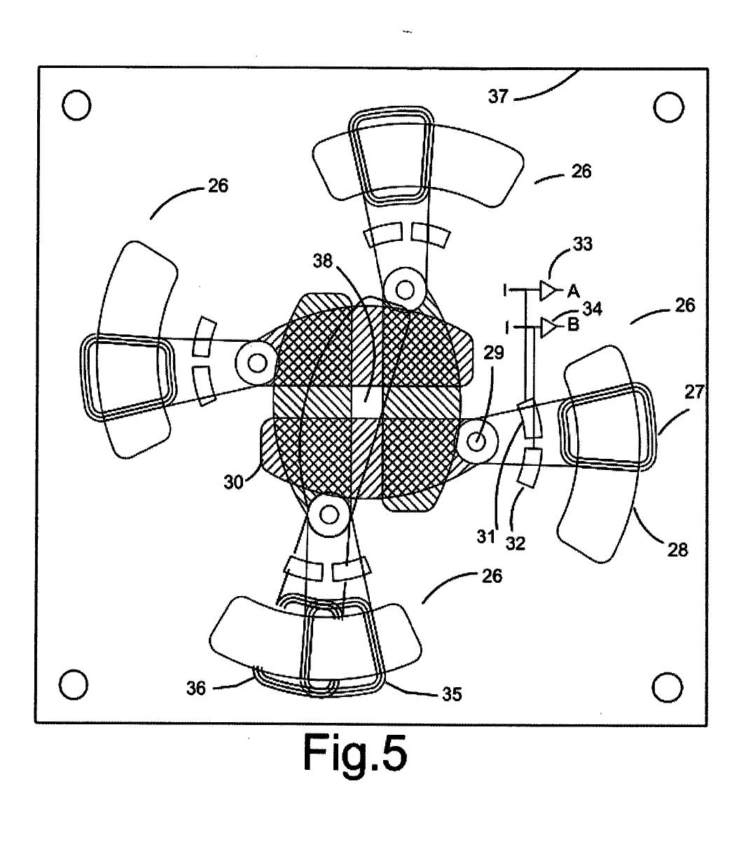

FIG. 5 shows a shutter mechanism based on such actuators. A plurality of actuators 26 are mounted on plate 37. The number of actuators can be from 3 to over 10 with the preferred number being 4, 6 or 8 units. Each actuator 26 controls a blade 30 made from X-ray absorbing material such as lead. It may be desirable to laminate the lead to a stiffer material such as thin stainless steel or aluminum. By the way of example, a 1 mm lead sheet can be bonded by soldering to a 0.4 mm spring tempered stainless steel sheet. The actuator comprises of a moving coil 27 pivoting on bearing 29 inside a magnetic field created by a permanent magnet 28. It is desirable to add a position sensor to the actuator, as it has to operate as part of a servo system under the control of the Shutter Control unit. A suitable sensor is a differential capacitance sensor comprising two electrodes 31 and 32 placed above the pivoting part without touching it. A typical gap between the electrodes and the moving part is 0.1-0.5 mm. It is assumed that the whole actuator is electrically grounded, therefore the capacitance from each electrode to ground is measured by monitoring the AC impedance, which is inversely proportional to the capacitance. If a constant AC current I is fed to the electrodes, the voltage, which is proportional to the impedance, can be sensed by amplifiers 33 and 34, creating a signal A and B inversely proportional to the overlap area. Such capacitive sensors are well known. The position of the pivoting arm is determined by the formula: (1/A−1/B)/(1/A+1/B) which equals to B−A/A+B. This ratio eliminates any dependence on amplitude of frequency stability. Typical values for the current are 10 uA to 1 mA at a frequency of 100 KHz to 1 MHz. Other position sensor such as optical encoders or inductive encoders can be used as well. The motion of the blade forming the aperture 38 is shown by the coil moving from position 35 to position 36, closing the aperture 38 completely. The aperture is not a square but an arbitrary shaped four sided polygon. More regular shapes can be achieved by more blades or by using rectilinear actuators. Since the software controls the blade position the shape of the arbitrary polygon is known and, if desired, the ROI image can be trimmed to a rectangle. If a true rectangular aperture is desired rectilinear actuators can be used or the rotary motion of the actuator can be converted to linear. Another solution is tilting plates in the style of venetian blinds. The shutter configuration of FIG. 5 requires only a space of a few mm between the collimator and the X-ray tube (the thickness of the blades), as the actuators can be places outside the flange connecting the tube to the collimator.

What is claimed is:

1. An X-Ray imaging system for displaying an image on a monitor comprising means for selecting an region of interest in said image and exposing said region of interest to a higher radiation dose than the rest of the image by using a plurality of X-Ray pulses for creating at least some of the images, the higher dose pulses used for the region of interest, using a high speed shutter to limit the high dose radiation to the region of interest, and combining the region of interest area and the rest of the image into a single image.

2. A system as in claim 1 wherein said region of interest is selected automatically by the system.

3. A system as in claim 1 wherein said region of interest is selected manually by the user.

4. A system as in claim 1 wherein said shutter comprises of at least four actuators, each one carrying an X-Ray absorbing blade and each one capable of being independently controlled.

5. A system as in claim 1 wherein motion of said shutter is synchronized to said X-Ray pulses.

6. A system as in claim 1 wherein said radiation dose is controlled by varying the length of said X-Ray pulses.

7. A system as in claim 1 wherein said radiation dose is controlled by varying the current to the X-Ray tube generating said X-Ray pulses.

8. A system as in claim 1 wherein both the location and shape are automatically selected and said selection can be over-ridden manually.

9. A method for X-ray imaging comprising the following steps:
controlling a shutter to select a region of interest in an X-Ray image, said region of interest being smaller than the desired image;
generating a high radiation dose X-Ray pulse;
opening said shutter to the size of the full image;
generating a lower dose X-Ray pulse; and
combining the high and low dose images into a single image.

10. A method for X-ray imaging as in claim 9 wherein said region of interest is automatically selected based on the data of said image.

11. A method for X-ray imaging as in claim 9 wherein said region of interest is selected manually.

12. A method for X-ray imaging as in claim 9 wherein both size and location of said region of interest are automatically selected but selection can be modified manually.

13. A system as in claim 1 wherein said region of interest is selected automatically by the system based on identifying a tool inserted into the body of the patient.

14. A method for X-ray imaging as in claim 9 wherein the region of interest is selected automatically by the system based on identifying a tool inserted into the body of the patient.

15. An X-Ray imaging system wherein at least some of the displayed images are formed by combining two images created by two X-ray pulses, a higher radiation dose pulse for imaging a region of interest and a lower radiation dose pulse for imaging the rest of the image.

16. A system as in claim 15 wherein said region of interest is defined by at least four X-Ray absorbing blades, the position of each blade individually controlled by the system.

17. A system as in claim 1 wherein the position and size of the region of interest can be controlled by a gesture based interface.

18. A system as in claim 15 wherein the position and size of the region of interest can be controlled by a gesture based interface.

19. A method for X-Ray imaging as in claim 9 wherein the position and size of the region of interest can be controlled by a gesture based interface.
